(19) United States
(12) Patent Application Publication
(10) Pub. No.: US 2006/0189645 A1
(43) Pub. Date: Aug. 24, 2006

(54) Novel 6-phenylphenantridines

(75) Inventor: Beate Schmidt, Allensbach (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 10/565,525

(22) PCT Filed: Jul. 30, 2004
(86) PCT No.: PCT/EP04/51679

§ 371 Date: Jan. 23, 2006

(30) Foreign Application Priority Data
Jul. 31, 2003 (EP) 03017337.1

(51) Int. Cl. C07D 221/12; A61K 31/473

(52) U.S. Cl. 514/298; 546/109

(57) ABSTRACT

Compounds of formula (I), in which R1, R2, R3, R31, R4, R5, R51, R6, R7 and R8 have the meanings indicated in the description, are novel effective PDE4 inhibitors.

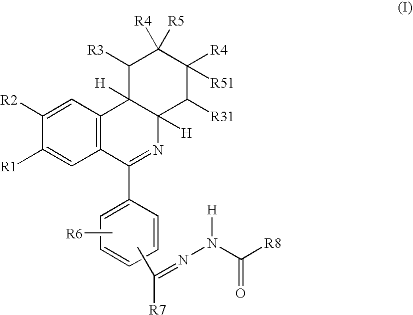

### FIELD OF APPLICATION OF THE INVENTION

The invention relates to novel 6-phenylphenanthridines, which are used in the pharmaceutical industry for the production of pharmaceutical compositions.

### KNOWN TECHNICAL BACKGROUND

The international applications WO 97/28131 (=U.S. Pat. No. 6,191,138), WO 97/35854 (=U.S. Pat. No. 6,127,378), WO 99/05113 (=U.S. Pat. No. 6,121,279), WO99/05111 (=U.S. Pat. No. 6,410,551), WO 00/42018, WO 00/42020, WO 02/05616 and WO 02/06238 describe 6-phenylphenanthridines as PDE4 inhibitors.

### DESCRIPTION OF THE INVENTION

It has now been found that the novel 6-phenylphenanthridines, which are described in greater detail below and differ from the previously known 6-phenylphenanthridines by unanticipated and sophisticated substitution patterns on the 6-phenyl ring, have surprising and particularly advantageous properties.

The invention thus relates to compounds of the formula I, 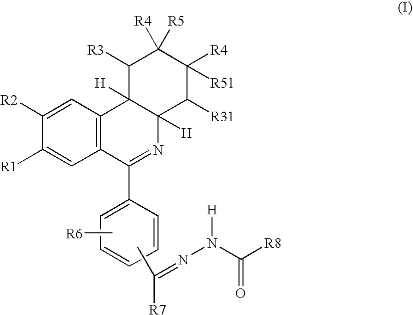
in which
R1 is hydroxyl, 1-4C-alkoxy, 3-7C-cycloalkoxy, 3-7C-cycloalkylmethoxy or completely or predominantly fluorine-substituted 1-4C-alkoxy,
R2 is hydroxyl, 1-4C-alkoxy, 3-7C-cycloalkoxy, 3-7C-cycloalkylmethoxy or completely or predominantly fluorine-substituted 1-4C-alkoxy,
   or in which R1 and R2 together are a 1-2C-alkylenedioxy group,
R3 is hydrogen or 1-4C-alkyl,
R31 is hydrogen or 1-4C-alkyl,
   or in which R3 and R31 together are a 1-4C alkylene group,
R4 is hydrogen or 1-4C-alkyl,
R5 is hydrogen,
R51 is hydrogen,
   or in which R5 and R51 together represent an additional bond.
R6 is hydrogen, halogen, nitro, 1-4C-alkyl, trifluoromethyl or 1-4-C-alkoxy,
R7 is 1-4C-alkyl, 3-7C-cycloalkyl, 3-7C-cycloalkylmethyl, pyridinyl, phenyl or R71- and/or R72-substituted phenyl, wherein
R71 is halogen, hydroxyl, cyano, trifluoromethyl, carboxyl, nitro, 1-4C-alkyl or 1-4C-alkoxy,
R72 is 1-4C-alkoxy,
R8 is 1-4C-alkyl, hydroxy-1-4C-alkyl, 1-4C-alkoxy-1-4C-alkyl, amino, C(O)N(H)R9, phenyl, HetA, aryl-1-4C-alkyl, HetB-1-4C-alkyl, cyano-1-4C-alkyl, 1-4C-alkoxycarbonyl-1-4C-alkyl or R14- and/or R15- and/or R16-substituted phenyl, wherein
R9 is hydrogen, phenyl or R91- and/or R92-substituted phenyl, wherein
R91 is halogen, 1-4C-alkyl, 1-4C-alkoxy, nitro, trifluoromethyl or completely or predominantly fluorine-substituted 1-4C-alkoxy,
R92 is halogen or 1-4C-alkoxy,
HetA is an unsubstituted or R10- and/or R11-substituted heteroaryl radical which is selected from the group consisting of pyrrolyl, furanyl, thiophenyl, pyrazolyl, imidazolyl, oxazolyl, isoxazolyl, thiazolyl, isothiazolyl, thiadiazolyl, pyridinyl, pyrimidinyl, pyrazinyl and pyridazinyl, wherein
R10 is 1-4C-alkyl, phenyl, halogen or trifluoromethyl,
R11 is 1-4C-alkyl,
aryl is phenyl or R12- and/or R13-substituted phenyl, wherein
R12 is 1-4C-alkyl, 1-4C-alkoxy, halogen, nitro or hydroxyl,
R13 is 1-4C-alkoxy or halogen,
HetB is an unsubstituted or R12- and/or R13-substituted indolyl radical,
R14 is 1-4C-alkyl, trifluoromethyl, 1-4C-alkoxy, halogen, nitro, hydroxyl, amino, mono- or di-1-4C-alkylamino or completely or predominantly fluorine-substituted 1-4C-alkoxy,
R15 is 1-4C-alkyl, 1-4C-alkoxy, halogen or completely or predominantly fluorine-substituted 1-4C-alkoxy,
R16 is 1-4C-alkoxy,
   and the salts and the E/Z isomers of these compounds.

1-4C-Alkyl represents a straight-chain or branched alkyl radical having 1 to 4 carbon atoms. Examples which may be mentioned are the butyl, isobutyl, sec-butyl, tert-butyl, propyl, isopropyl and preferably the ethyl and methyl radicals.

1-4C-Alkoxy represents radicals which, in addition to the oxygen atom, contain a straight-chain or branched alkyl radical having 1 to 4 carbon atoms. Examples which may be mentioned are the butoxy, isobutoxy, sec-butoxy, tert-butoxy, propoxy, isopropoxy and preferably the ethoxy and methoxy radicals.

3-7C-Cycloalkoxy represents cyclopropyloxy, cyclobutyloxy, cyclopentyloxy, cyclohexyloxy and cycloheptyloxy, of which cyclopropyloxy, cyclobutyloxy and cyclopentyloxy are preferred.

3-7C-Cycloalkylmethoxy represents cyclopropylmethoxy, cyclobutylmethoxy, cyclopentylmethoxy, cyclohexylmethoxy and cycloheptylmethoxy, of which cyclopropylmethoxy, cyclobutylmethoxy and cyclopentylmethoxy are preferred.

As completely or predominantly fluorine-substituted 1-4C-alkoxy, for example, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy radicals may be mentioned. “Predominantly” in this connection means that more than half of the hydrogen atoms of the 1-4C-alkoxy radicals are replaced by fluorine atoms.

1-2C-Alkylenedioxy represents, for example, the methylenedioxy [—O—CH₂—O—] and the ethylenedioxy [—O—CH₂—CH₂—O—] radicals.

If R3 and R31 together have the meaning 1-4C-alkylene, the positions 1 and 4 in compounds of the formula I are linked to one another by a 1-4C-alkylene bridge, 1-4C-alkylene representing straight-chain or branched alkylene radicals having 1 to 4 carbon atoms. Examples which may be mentioned are the radicals methylene [—CH₂—], ethylene [—CH₂—CH₂—], trimethylene [—CH₂—CH₂CH₂—], 1,2-dimethylethylene [—CH(CH₃)—CH(CH₃)—] and isopropylidene [—C(CH₃)₂—].

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkylmethyl represents a methyl radical which is substituted by one of the abovementioned 3-7C-cycloalkyl radicals. Preferably, the 3-5C-cycloalkylmethyl radicals cyclopropylmethyl, cyclobutylmethyl and cyclopentylmethyl may be mentioned.

1-4C-Alkoxycarbonyl represents a carbonyl group to which one of the abovementioned 1-4C-alkoxy radicals is bonded. Examples which may be mentioned are the methoxycarbonyl [CH₃O—C(O)—] and the ethoxycarbonyl [CH₃CH₂O—C(O)—] radicals.

Hydroxy-1-4C-alkyl represents abovementioned 1-4C-alkyl radicals, which are substituted by a hydroxyl group. Examples which may be mentioned are the 2-hydroxyethyl and the 3-hydroxypropyl radicals.

1-4C-Alkoxy-1-4C-alkyl represents one of the abovementioned 1-4C-alkyl radicals, which is substituted by one of the abovementioned 1-4C-alkoxy radicals. Examples which may be mentioned are the methoxymethyl, the 2-methoxyethyl and the 3-methoxypropyl radicals.

HetA represents an unsubstituted or R10- and/or R11-substituted heteroaryl radical which is selected from the group consisting of pyrrolyl, furanyl, thiophenyl, pyrazolyl, imidazolyl, oxazolyl, isoxazolyl, thiazolyl, isothiazolyl, thiadiazolyl, pyridinyl, pyrimidinyl, pyrazinyl and pyridazinyl.

Exemplary unsubstituted heteroaryl radicals HetA which may be mentioned are furan-2-yl, furan-3-yl, thiophen-2-yl, thiophen-3-yl, 1H-pyrrol-2-yl, 1H-pyrrol-3-yl, pyrazol-3-yl, pyrazol-4yl, imidazol-2-yl imidazol-4-yl, imidazol-5-yl, oxazol-2-yl, oxazol-4-yl, oxazol-5-yl, thiazol-2-yl, thiazol-4-yl, thiazol-5-yl isoxazol-4-yl, isoxazol-5-yl, isothiazol-3-yl, isothiazol-4-yl, isothiazol-5-yl, [1,2,3]thiadiazol-4yl, [1,2,3]thiadiazol-5-yl, pyridin-2-yl, pyridin-3-yl, pyridin-4-yl, pyrimidin-4-yl, pyrimidin-5-yl, pyrimidin-2-yl, pyridazin-4-yl, pyridazin-3-yl and pyrazin-2-yl.

Exemplary R10- and/or R11-substituted heteroaryl radicals HetA which may be mentioned are 1-methyl-1H-pyrrol-2-yl, 5-methyl-3-phenylisoxazol-4-yl, 5-methylthiophen-2-yl, 3-methyl-furan-2-yl, 3,5-dimethylisoxazol-4-yl, 4-phenyl-[1,2,3]thiadiazol-5-yl, 4-methyl[1,2,3]thiadiazol-5-yl, 1,5-dimethyl-1H-pyrazol-3-yl, 3-methyl-1H-pyrazol-5-yl, 2-chloro-6-methylpyrimidin-4-yl, 5-methylpyrazin-2-yl, 2-methylpyrazin-5-yl and 5-chloropyrazin-2-yl.

Aryl stands for phenyl or R12- and/or R13-substituted phenyl.

Aryl-1-4C-alkyl represents one of the abovementioned, aryl-substituted 1-4C-alkyl radicals, wherein aryl has the abovementioned meanings. Examples which may be mentioned are the 2-arylethyl and, preferably, the arylmethyl radicals.

HetB represents an unsubstituted or R12- and/or R13-substituted indolyl radical. Preferred indolyl radicals are the indol-2-yl and, in particular, the indol-3-yl radicals. A R12- and/or R13-substituted indolyl radical is preferably substituted on the benzo ring.

HetB-1-4C-alkyl represents one of the abovementioned 1-4C-alkyl radicals which is substituted by one of the abovementioned HetB radicals. Examples which may be mentioned are the HetB-ethyl and, preferably, the HetB-methyl radicals.

Cyano-1-4C-alkyl represents one of the abovementioned 1-4C-alkyl radicals which is substituted by a cyano radical. Examples which may be mentioned are the 2-cyanoethyl and, preferably, the cyanomethyl radicals.

1-4C-Alkoxycarbonyl-1-4C-alkyl represents one of the abovementioned 1-4C-alkyl radicals which is substituted by one of the abovementioned 1-4C-alkoxycarbonyl radicals. Examples which may be mentioned are the 1-4C-alkoxycarbonylethyl and, preferably, the 1-4C-alkoxycarbonylmethyl radicals.

In addition to the nitrogen atom, mono- or di-1-4C-alkylamino radicals contain one or two of the abovementioned 1-4C-alkyl radicals. Di-1-4C-alkylamino is preferred and here, in particular, dimethyl-, diethyl- or diisopropylamino.

Halogen within the meaning of this invention is bromine, chlorine or fluorine.

Pyridinyl within the meaning of this invention is pyridin-2-yl, pyridin-3-yl or pyridin-4-yl.

The substituents R6 and —C(R7)=N—N(H)—C(O)R8 of compounds of the formula I can be attached in the ortho, meta or para position with respect to the binding position in which the 6-phenyl ring is bonded to the phenanthridine ring system. Preference is given to compounds of the formula I, in which R6 is hydrogen and —C(R7)=N—N(H)—C(O)R8 is attached in the meta or in the para position.

The person skilled in the art knows that compounds comprising a non-ring C═N double bond can exist in two stereoisomeric forms denoted according common practice in stereochemistry as Z/E isomers. With respect to the hydrazone C═N double bond, the invention thus relates to any of the possible Z/E isomers and mixtures thereof.

Possible salts for compounds of the formula I-depending on substitution—are all acid addition salts or all salts with bases. Particular mention may be made of the pharmacologically tolerable salts of the inorganic and organic acids and bases customarily used in pharmacy. Those suitable are, on the one hand, water-insoluble and, particularly, water-soluble acid addition salts with acids such as, for example, hydrochloric acid, hydrobromic acid, phosphoric acid, nitric acid, sulfuric add, acetic add, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic add, oxalic acid, tartaric acid, embonic acid, stearic acid, toluenesulfonic acid, methanesulfonic acid or 3-hydroxy-2-naphthoic acid, it being possible to employ the acids in salt preparation—depending on whether a mono- or polybasic acid is concerned and depending on which salt is desired—in an equimolar quantitative ratio or one differing therefrom.

On the other hand, salts with bases are also suitable. Examples of salts with bases which may be mentioned are alkali metal (lithium, sodium, potassium) or calcium, aluminum, magnesium, titanium, ammonium, meglumine or guanidinium salts, where here too the bases are employed in salt preparation in an equimolar quantitative ratio or one differing therefrom.

Pharmacologically intolerable salts which can initially be obtained, for example, as process products in the preparation of the compounds according to the invention on an industrial scale are converted into pharmacologically tolerable salts by processes known to the person skilled in the art.

It is known to the person skilled in the art that the compounds according to the invention and their salts, when they are isolated, for example, in crystalline form, can contain various amounts of solvents. The invention therefore also comprises all solvates and in particular all hydrates of the compounds of the formula I, and also all solvates and in particular all hydrates of the salts of the compounds of the formula I.

Compounds of the formula I to be emphasized are those in which
R1 is 1-2C-alkoxy, 3-5C-cycloalkoxy, 3-5C-cycloalkylmethoxy or completely or predominantly fluorine-substituted 1-2C-alkoxy,
R2 is 1-2C-alkoxy, 3-5C-cycloalkoxy, 3-5C-cycloalkylmethoxy or completely or predominantly fluorine-substituted 1-2C-alkoxy,
R3 is hydrogen,
R31 is hydrogen,
R4 is hydrogen or 1-2C-alkyl,
R5 is hydrogen,
R51 is hydrogen,
   or in which R5 and R51 together represent an additional bond,
R6 is hydrogen,
R7 is 1-4C-alkyl or phenyl, R8 is 1-4C-alkyl, hydroxy-2-4C-alkyl, amino, C(O)N(H)R9, phenyl, HetA, aryl-1-2C-alkyl, HetB-1-2C-alkyl, cyano-1-2C-alkyl, 1-4C-alkoxycarbonyl-1-2C-alkyl or R14- and/or R15- and/or R16-substituted phenyl, wherein
R9 is hydrogen, phenyl or R91-substituted phenyl, wherein
R91 is halogen,
HetA is an unsubstituted or R10-substituted heteroaryl radical which is selected from the group consisting of pyrrolyl, furanyl, pyrazolyl, thiadiazolyl and pyridinyl, wherein
R10 is 1-4C-alkyl,
aryl is phenyl or R12- and/or R13-substituted phenyl, wherein
R12 is 1-4C-alkoxy,
R13 is 1-4C-alkoxy,
HetB is an unsubstituted or R12- and/or R13-substituted indol-3-yl radical,
R14 is 1-4C-alkyl, trifluoromethyl, 1-4C-alkoxy, halogen, nitro, hydroxyl, amino, mono- or di-1-4C-alkylamino or completely or predominantly fluorine-substituted 1-2C-alkoxy,
R15 is 1-4C-alkoxy or completely or predominantly fluorine-substituted 1-2C-alkoxy,
R16 is 1-4C-alkoxy,
   and the salts and the E/Z isomers of these compounds.

Compounds of the formula I to be more emphasized are those in which
R1 is methoxy,
R2 is methoxy,
R3, R31, R4, R5 and R51 are hydrogen,
R6 is hydrogen,
R7 is methyl or phenyl,
R8 is methyl, hydroxypropyl, amino, C(O)N(H)R9, phenyl, HetA, arylmethyl, indol-3-ylmethyl, cyanomethyl, ethoxycarbonylmethyl or R14- and/or R15- and/or R16-substituted phenyl, wherein
R9 is hydrogen or R91-substituted phenyl, wherein
R91 is fluorine,
HetA is an unsubstituted furanyl, pyrrolyl or pyridinyl radical, a R10-substituted thiadiazolyl radical or a R10-substituted pyrazolyl radical, wherein
R10 is methyl,
aryl is phenyl or R12- and/or R13-substituted phenyl, wherein
R12 is methoxy,
R13 is methoxy,
R14 is methyl, trifluoromethyl, methoxy, fluoro, chloro, nitro, hydroxyl, amino or dimethylamino,
R15 is methoxy,
R16 is methoxy,
   and the salts and the E/Z isomers of these compounds.

Compounds of the formula I to be in particular emphasized are those in which either
R1 is methoxy,
R2 is methoxy,
R3, R31, R4, R5 and R51 are hydrogen,
R6 is hydrogen,
R7 is methyl,
R8 is methyl, hydroxypropyl, amino, C(O)N(H)R9, phenyl, HetA, 3-methoxyphenyl, 4-chlorophenyl, 4-nitrophenyl, 4-hydroxyphenyl, 4-aminophenyl, 4-methylphenyl, 3-chlorophenyl, 3-nitrophenyl, 3,4-dimethoxyphenyl, 3,4,5-trimethoxyphenyl, 4-dimethylaminophenyl, 4-methoxyphenyl, 4-trifluoromethylphenyl, 4-fluorophenyl, arylmethyl, indol-3-ylmethyl, cyanomethyl or ethoxycarbonylmethyl, wherein
R9 is hydrogen or 4-fluorophenyl,
HetA is 1H-pyrrol-2-yl, pyridin-4-yl, furan-2-yl, pyridin-3-yl, 3-methyl-1H-pyrazol-5-yl or 4-methyl[1,2,3]thiadiazol-5-yl,
aryl is phenyl, 4-methoxyphenyl or 3,4-dimethoxyphenyl,
   or
R1 is methoxy,
R2 is methoxy,
R3, R31, R4, R5 and R51 are hydrogen,
R6 is hydrogen,
R7 is phenyl,
R8 is methyl or amino,
   and the salts and the E/Z isomers of these compounds.

A special embodiment of the compounds of the present invention include those compounds of the formula I in which R1 and R2 are 1-2C-alkoxy.

Another special embodiment of the compounds of the present invention include those compounds of the formula I in which R1 and R2 are 1-2C-alkoxy and R3, R31, R4, R5 and R51 are hydrogen.

A further special embodiment of the compounds of the present invention include those compounds of the formula I in which R1and R2 are 1-2C-alkoxy and R3, R31, R4, R5, R51 and R6 are hydrogen.

Still a further special embodiment of the compounds of the present invention include those compounds of the formula I in which R1 and R2 are 1-2C-alkoxy and R3, R31, R4, R5, R51 and R6 are hydrogen and R7 is methyl.

Still a further special embodiment of the compounds of the present invention include those compounds of the formula I in which R1 and R2 are 1-2C-alkoxy and R3, R31, R4, R5, R51 and R6 are hydrogen and R7 is phenyl.

The compounds of the formula I are chiral compounds having chiral centers at least in positions 4a and 10b and, depending on the meaning of the substituents R3, R31, R4, R5 and R51, further chiral centers in the positions 1, 2, 3 and 4.

Numbering:

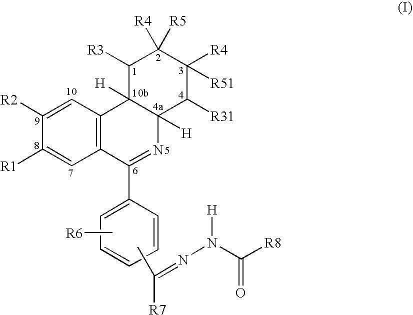

The invention therefore comprises all conceivable stereoisomers in pure form as well as in any mixing ratio.

Preferred compounds of the formula I are those in which the hydrogen atoms in positions 4a and 10b are in the cis position relative to one another. The pure cis diastereomers, the pure cis enantiomers and their mixtures in any mixing ratio and including the racemates are more preferred in this context. Particularly preferred in this connection are those compounds of the formula I which have, with respect to the positions 4a and 10b, the same configuration as shown in the formula I*:

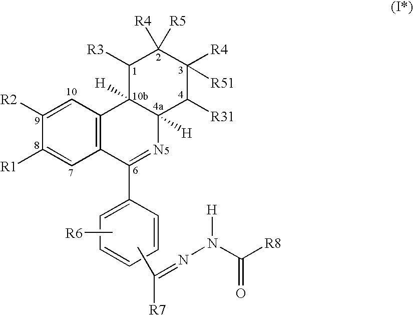

If, for example in compounds of the formula I* R3, R31, R4, R5 and R51 have the meaning hydrogen, then the configuration—according the rules of Cahn, Ingold and Prelog—is R in the position 4a and R in the position 10b.

The enantiomers can be separated in a manner known per se (for example by preparation and separation of appropriate diastereoisomeric compounds). For example, an enantiomer separation can be carried out at the stage of the starting compounds of the formula V in which R1, R2, R3, R31, R4, R5 and R51 have the meanings indicated above.

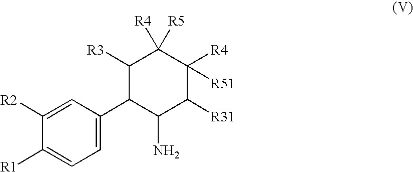

Separation of the enantiomers can be carried out, for example, by means of salt formation of the racemic compounds of the formula V with optically active acids, preferably carboxylic adds, subsequent resolution of the salts and release of the desired compound from the salt. Examples of optically active carboxylic acids which may be mentioned in this connection are the enantiomeric forms of mandelic acid, tartaric acid, O,O′-dibenzoyltartaric acid, camphoric acid, quinic acid, glutamic acid, malic acid, camphorsulfonic acid, 3-bromocamphorsulfonic acid, α-methoxyphenylacetic acid, α-methoxy-α-trifluoromethylphenylacetic acid and 2-phenylpropionic acid. Alternatively, enantiomerically pure starting compounds of the formula V can be prepared via asymmetric syntheses. Enantiomerically pure starting compounds as well as enantiomerically pure compounds of the formula I can be also obtained by chromatographic separation on chiral separating columns; by derivatization with chiral auxiliary reagents, subsequent diastereomer separation and removal of the chiral auxiliary group; or by (fractional) crystalliation from a suitable solvent.

The compounds according to the invention can be prepared, for example, according to the subsequently specified reaction steps shown in reaction schemes 1 and 2.

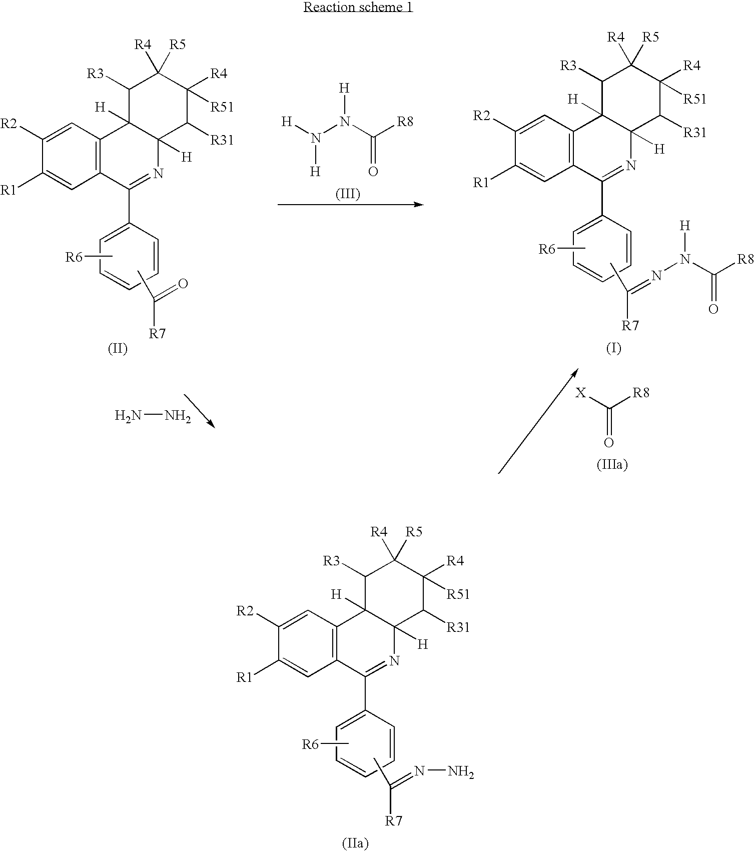

Reaction scheme 1 shows by way of example two alterative synthesis routes for compounds of the formula I, in which R1, R2, R3, R31, R4, R5, R51, R6, R7 and R8 have the meanings indicated above, starting from keto compounds of the formula II, in which R1, R2, R3, R31, R4, R5, R51, R6 and R7 have the meanings indicated above.

One the one hand, said compounds of the formula I are accessible by acylhydrazone formation reaction of said compounds of the formula II with compounds of the formula III, in which R8 has the said meaning. Said reaction, can be carried out, for example, as described in the following examples or in a manner known to one of ordinary skill in the art.

On the other hand, said compounds of the formula I can be also obtained in a two step procedure starting from said compounds of the formula II: Firstly, compounds of the formula II are converted with hydrazine by hydrazone formation reaction into corresponding compounds of the formula IIa and then, compounds of the formula IIa obtained are reacted with compounds of the formula IIIa, in which R8 has the meanings indicated above and X represents a suitable leaving group, for example a chlorine atom or an acyloxy leaving group, to obtain in an acylation reaction the desired compounds of the formula I. Both reactions mentioned above can be carried out as known to the person skilled in the art.

Compounds of the formula III are either commercially available or can be prepared in an art-known manner.

Compounds of the formula IIIa are known or can be prepared according to known procedures.

Compounds of the formula II, in which R1, R2, R3, R31, R4, R5, R51, R6 and R7 have the meanings indicated above, are either known from the international application WO00/42020 or can be prepared similarly or analogously as described herein. Preferably, however, compounds of the formula II are obtained according to those procedures given by way of example in the following examples. For greater detail, a suitable synthesis route for compounds of the formula II is outlined in reaction scheme 2 below. In the first step of said reaction scheme 2 compounds of the formula V, in which R1, R2, R3, R31, R4, R5 and R51 have the meanings given above, are reacted with compounds of the formula VI, in which R6 and R7 have the meanings given above and X represents a suitable leaving group, preferably a chlorine atom, to give compounds of the formula IV, in which R1, R2, R3, R31, R4, R5, R51, R6 and R7 have the abovementioned meanings.

Alternatively, compounds of the formula IV, in which R1, R2, R3, R31, R4, R5, R51, R6 and R7 have the meanings given above, can also be prepared, for example, from compounds of the formula V, in which R1, R2, R3, R31, R4, R5 and R51 have the abovementioned meanings, and compounds of the formula VI, in which R6 and R7 have the abovementioned meanings and X is hydroxyl, by reaction with amide bond linking reagents known to the person skilled in the art. Exemplary amide bond linking reagents known to the person skilled in the art which may be mentioned are, for example, the carbodiimides (e.g. dicyclohexylcarbodiimide or, preferably, 1-ethyl-3-(3-dimethylaminopropyl)carbodiimide hydrochloride), azodicarboxylic acid derivatives (e.g. diethyl azodicarboxylate), uronium salts [e.g. O-(benzotriazol-1-yl)-N,N,N′,N′-tetramethyluronium tetrafluoroborate or O-(benzotriazol-1yl)-N,N,N′,N′-tetramthyl-uronium-hexafluorophosphate] and N,N′-carbonyldiimidazole. In the scope of this invention preferred amide bond linking reagents are uronium salts and, particularly, carbodiimides, preferably, 1-ethyl-3-(3-dimethylaminopropyl)carbodiimide hydrochloride.

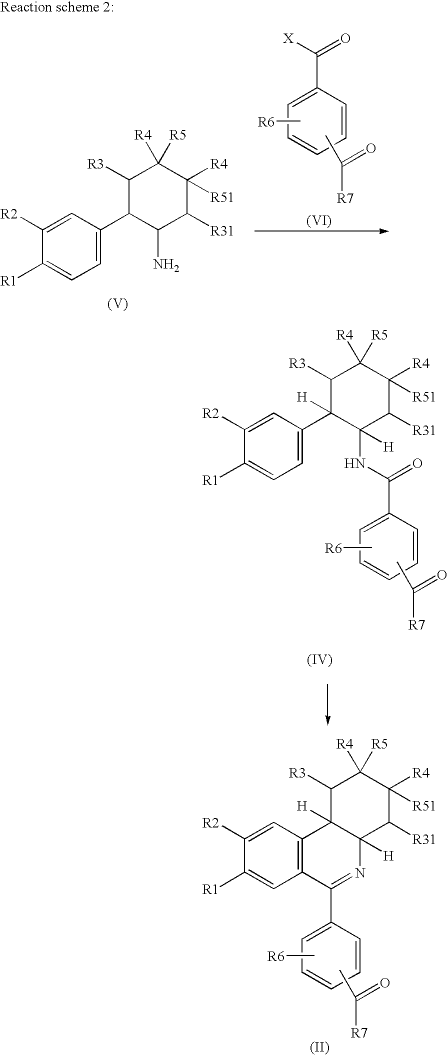

Compounds of the formula VI, wherein R6 and R7 have the abovementioned meanings, are either known or can be prepared in a known manner.

As shown in the next step within reaction scheme 2, compounds of the formula II, in which R1, R2, R3, R31, R4, R5, R51, R6 and R7 have the meanings indicated above, can be obtained by cyclocondensation of corresponding compounds of the formula IV. Said cyclocondensation reaction is carried out in a manner habitual per se to the person skilled in the art or as described by way of example in the following examples, according to Bischler-Napieralski (e.g. as described in J. Chem. Soc., 1956, 4280-4282) in the presence of a suitable condensing agent, such as, for example, polyphosphoric acid, phosphorus pentachloride, phosphorus pentoxide or phosphorus oxychloride, in a suitable inert solvent, e.g. in a chlorinated hydrocarbon such as chloroform, or in a cyclic hydrocarbon such as toluene or xylene, or another inert solvent such as acetonitrile, or without further solvent using an excess of condensing agent, at reduced temperature, or at room temperature, or at elevated temperature or at the boiling temperature of the solvent or condensing agent used.

The preparation of pure enantiomeres of starting compounds of the formula V can be carried out as described, for example, in the international application WO00/42020 or in a manner according to the following examples.

It is moreover known to the person skilled in the art that if there are a number of reactive centers on a starting or intermediate compound it may be necessary to block one or more reactive centers temporarily by protective groups in order to allow a reaction to proceed specifically at the desired reaction center. A detailed description for the use of a large number of proven protective groups is found, for example, in “Protective Groups in Organic Synthesise” by T. Greene and P. Wuts (John Wiley & Sons, Inc. 1999, 3^{rd} Ed.) or in “Protecting Groups (Thieme Foundations Organic Chemistry Series N Group” by P. Kocienski (Thieme Medical Publishers, 2000).

The isolation and purification of the substances according to the invention is carried out in a manner known per se, e.g. by distilling off the solvent in vacuo and recrystallizing the resulting residue from a suitable solvent or subjecting it to one of the customary purification methods, such as, for example, column chromatography on suitable support material.

Salts are obtained by dissolving the free compound in a suitable solvent (e.g. a ketone, such as acetone, methyl ethyl ketone or methyl isobutyl ketone, an ether, such as diethyl ether, tetrahydrofuran or dioxane, a chlorinated hydrocarbon, such as methylene chloride or chloroform, or a low molecular weight aliphatic alcohol such as ethanol or isopropanol) which contains the desired acid or base, or to which the desired acid or base is then added. The salts are obtained by filtering, reprecipitating, precipitating with a nonsolvent for the addition salt or by evaporating the solvent. Salts obtained can be converted by alkalization or by acidification into the free compounds, which in turn can be converted into salts. In this way, pharmacologically intolerable salts can be converted into pharmacologically tolerable salts.

Optionally, compounds of the formula I can be converted into their salts, or, optionally, salts of the compounds of the formula I can be converted into the free compounds.

The person skilled in the art knows on the basis of his/her knowledge and on the basis of those synthesis routes, which are shown and described within the description of this invention, how to find other possible synthesis routes for compounds of the formula I. All these other possible synthesis routes are also part of this invention.

The following examples serve to illustrate the invention in greater detail without restricting it. Likewise, further compounds of the formula I, whose preparation is not explicitly described, can also be prepared in an analogous manner or in a manner familiar per se to the person skilled in the art using customary process techniques.

In the examples, m.p. stands for melting point, h for hour(s), min for minutes, conc. for concentrated, satd. for saturated, EF for empirical formula, MW for molecular weight, MS for mass spectrum, M for molecular ion, fnd. for found, calc. for calculated.

The compounds mentioned in the examples and their salts and E/Z isomers are a preferred subject of the invention.

### EXAMPLES

Final products:

### 1. Nicotinic acid {1-[4-((4aR,10bR)-8,9-dimethoxy-1,2,3,4,4a,10b-hexahydro-phenanthridin-6-yl)-phenyl]-ethylidene}-hydrazide

200 mg of (4aR,10bR)-8,9-Dimethoxy-6-(4-acetophenyl1,2,3,4,4a,10b-hexahydrophenanthridine (compound A1) and 400 mg of nicotinic acid hydrazide are refluxed in a mixture of 5 ml of absolute ethanol and 0.4 ml of glacial acetic acid for 2 h. Afterwards, the reaction solution is concentrated, the residue is redissolved in ethyl acetate and washed with saturated sodium hydrogencarbonate solution. The organic phase is dried using sodium sulfate, concentrated and the residue is treated with diethyl ether. The precipitate obtained is filtered off and dried. 170 mg of the title compound are obtained.

M.p.: 162° C.

MS: calc.: C₂₉H₃₀N₄O₃ (482.59) fnd.: [M+1] 483.3

Starting from the appropriate starting compounds A1 or A5 or A8 described below, the following compounds are obtained in analogy to the procedure as in Example 1 using appropriately substituted acylhydrazides as reaction partners.

### 2. Cyano-acetic acid {1-[4-((4aR,10bR)-8,9-dimethoxy-1,2,3,4,4a,10b-hexahydro-phenanthridin-6-yl)-phenyl]-ethylidene}-hydrazide

MS: calc.: C₂₆H₂₈N₄O₃ (444.54) fnd.: [M+1] 445.3

### 3. Isonicotinic acid {1-[4-((4aR,10bR)-8,9-dimethoxy-1,2,3,4,4a,10b-hexahydro-phenanthridin-6-yl)-phenyl]-ethylidene}-hydrazide

MS: calc.: C₂₉H₃₀N₄O₃ (482.59) fnd.: [M+1] 483.3

### 4. Acetic acid {1-[4-((4aR,10bR)-8,9-dimethoxy-1,2,3,4,4a,10b-hexahydro-phenanthridin-6-yl)-phenyl]-ethylidene}-hydrazide

MS: calc.: C₂₅H₂₉N₃O₃ (419.53) fnd.: [M+1] 420.3

### 5. Acetic acid {1-[4-((4aR,10bR)-8,9-dimethoxy-1,2,3,4,4a,10b-hexahydro-phenanthridin-6-yl)-phenyl]-ethylidene}-hydrazide

MS: calc.: C₃₀H₃₁N₃O₃ (481.6) fnd.: [M+1] 482.4

### 6. 1-[4-((4aR,10bR)-8,9-Dimethoxy-1,2,3,4,4a,10b-hexahydro-phenanthridin-6-yl)-phenyl]-ethanone-semicarbazone

MS: calc.: C₂₄H₂₈N₄O₃ (420.52) fnd.: [M+1] 421.3

### 7. 1-[4-((4aR,10bR)-8,9-Dimethoxy-1,2,3,4,4a,10b-hexahydro-phenanthridin-6-yl)-phenyl]-1-phenyl-methanone-semicarbazone

MS: calc.: C₂₉H₃₀N₄O₃ (482.59) fnd.: [M+1] 483.3

8. Acetic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₅H₂₈N₃O₃ (419.53) fnd.: [M+1] 420.4

### 9. Isonicotinic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₉H₃₀N₄O₃ (482.59) fnd.: [M+1] 483.1

### 10. Furan-2-carboxylic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₈H₂₉N₃O₄ (471.56) fnd.: [M+1] 472.1

### 11. (1H-indol-3-yl)-acetic acid {1-[3-((4aR10bR)-8,9-dimethoxy-1,2,3,4,4a,10b-hexahydrophenanthridin-6-yl)-phenyl]-ethylidene}-hydrazide

MS: calc.: C₃₃H₃₄N₄O₃ (534.66) fnd.: [M+1] 535.2

### 12. Benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₁N₃O₃ (481.6) fnd.: [M+1] 482.2

### 13. 3-Methoxy-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₁H₃₃N₃O₄ (511.63) fnd.: [M+1] 512.2

### 14. 4Chloro-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₀N₃O₃(516,04) fnd.: [M+1] 516,2

### 15. 4-Nitro-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₀N₄O₅ (526.6) fnd.: [M+1] 527.2

### 16. 4Hydroxy-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₁N₃O₄ (497.6) fnd.: [M+1] 498.2

### 17. 4-Amino-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₂N₄O₃ (496.61) fnd.: [M+1] 497.2

### 18. 4-Methyl-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₁H₃₃N₃O₃ (495.63) fnd.: [M+1] 496.2

### 19. Phenyl-acetic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₁H₃₃N₃O₃ (495.63) fnd.: [M+1] 496.2

### 20. 2-(N′-(1-(3-((4aR,10bR)-8,9-Dimethoxy-1,2,3,4,4a,10b-hexahydro-phenanthridin-6-yl)-phenyl]-ethylidene}-hydrazino)-2-oxo-acetamide

MS: calc.: C₂₅H₂₆N₄O₄ (448.53) fnd.: [M+1] 449.1

### 21. 3-Chloro-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₀ClN₃O₃ (516.04) fnd.: [M+1] 516.2

### 22. 3,4,5-Trimethoxy-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₃H₃₇N₃O₅ (571.68) fnd.: [M+1] 572.2

### 23. 4-Dimethylamino-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₂H₃₆N₄O₃ (524.67) fnd.: [M+1] 525.2

### 24. 3-Nitro-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₀N₄O₅ (526.6) fnd.: [M+1] 527.2

### 25. 3,4-Dimethoxy-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₂H₃₅N₃O₅ (541.65) fnd.: [M+1] 542.2

### 26. 4-Methoxy-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₁H₃₃N₃O₄ (511.63) fnd.: [M+1] 612.2

### 27. (3,4-Dimethoxy-phenyl)-acetic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₃H₃₇N₃O₅ (555.68) fnd.: [M+1] 556.2

### 28. 4-Hydroxy-butyric acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₇H₃₃N₃O₄ (463.58) fnd.: [M+1] 464.2

### 29. 4-Trifluoromethyl-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₁H₃₀F₃N₃O₃ (549.6) fnd.: [M+1] 550.2

### 30. 4-Methyl-[1,2,3]thiadiazole-5-carboxylic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₇H₂₈N₅O₃S (503,63) fnd.: [M+1] 504,0

### 31. 4-Fluoro-benzoic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₀H₃₀FN₄O₃ (499.59) fnd.: [M+1] 500.2

### 32. 5-Methyl-2H-pyrazole-3-carboxylic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₈H₃₁N₅O₃ (485.59) fnd.: [M+1] 486.2

### 33. (3-Methoxy-phenyl)-acetic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₃₂H₃₅N₃O₄ (525.65) fnd.: [M+1] 526.2

### 34. {1-[3-((4aR10bR)-8,9-dimethoxy-1,2,3,4,4a,10b-hexahydro-phenanthridin-6-yl)-phenyl]-ethylidene-hydrazinocarbonyl}-acetic acid ethyl ester

MS: calc.: C₂₈H₃₃N₃O₅ (491.59) fnd.: [M+1] 492.2

### 35. 1H-Pyrrole-2-carboxylic acid {1-[3-((4aR10bR)-8,9-dimethoxy-1,2,3,4,4a,10b-hexahydro-phenanthridin-6-yl)-phenyl]-ethylidene}-hydrazide

MS: calc.: C₂₈H₃₀N₄O₃ (470.58) fnd.: [M+1] 471.1

### 36. 2-(N′-{1-[3-((4aR10bR)-8,9-Dimethoxy-1,2,3,4,4a,10b-hexahydro-phenanthridin-6-yl)-phenyl]-ethylidene}-hydrazino)-N-(4-fluoro-phenyl)-2oxo-acetamide

MS: calc.: C₃₁H₃₁FN₄O₃ (542.62) fnd.: M+1] 543.1

### 37. 1-[3-((4aR10bR)-8,9-Dimethoxy-1,2,3,4,4a,10b-hexahydro-phenanthridin-6-yl)-phenyl]-ethanone-semicarbazone

MS: calc.: C₂₄H₂₈N₄O₃ (420.52) fnd.: [M+1] 421.2

Starting compounds:

### A1. (4aR,10bR)-8,9-Dimethoxy6-(4-acetophenyl)-1,2,3,4,4a,10b-hexahydrophenanthridine

Compound A1 is prepared from compound B1 analogously as described in Example A5.

EF: C₂₃H₂₅NO₃; MW: 363.46

Elemental analysis: calc.: C, 76.01; H, 6.93; N, 3.85.

fnd : C, 75.77; H, 6.98; N, 3.82.

Optical rotation: [α]_{D}²⁰=−97.4° (c=0.2, ethanol)

### A5. (4aR,10bR)-8,9-Dimethoxy-6-(4-benzoylphenyl)-1,2,3,4,4a,10b-hexahydrophenanthridine

7.1 g of (−)cis-N-[2-(3,4-Dimethoxyphenyl)cyclohexyl]-4-benzoylbenzamide (compound B5) are dissolved in 100 ml of acetonitrile and 5.0 ml of phosphoryl chloride and stirred overnight at 80° C. The reaction mixture is concentrated under reduced pressure and the residue is extracted with satd. sodium hydrogencarbonate solution and ethyl acetate. After chromatography on silica gel using petroleum ether (low)/ethyl acetate/triethylamine in the ratio 6/3/1 and concentration of the product fractions, 5.3 g of the title compound are obtained.

EF: C₂₈H₂₇NO₃; MW: 425.53

Elemental analysis x 0.08 H₂O: calc.: C, 78.77; H, 6.41; N, 3.28.

fnd: C, 78.55; H, 6.64; N, 3.50.

Optical rotation: [α]_{D}²⁰=−70.6° (c=0.2, ethanol)

### A8. (4aR,10bR)-8,9-Dimethoxy6-(3-acetophenyl)-1,2,3,4,4a,10b-hexahydrophenanthridine

Compound A8 is prepared from compound B8 analogously as described in Example A5.

M.p. 112.5-114° C.

EF: C₂₃H₂₅NO₃; MW: 363.46

Elemental analysis: calc.: C, 76.01; H, 6.93; N, 3.85.

fnd.: C, 75.62; H, 6.90; N, 3.83.

Optical rotation: [α]_{D}²⁰=−168.7° (c=0.2, ethanol)

### B1. N-[(1R,2R)-2-(3,4-Dimethoxyphenyl)cyclohexyl]-4-acetobenzamide

Compound B1 is prepared from compound C1 analogously as described in Example B5.

M.P.: 129-137° C.

Optical rotation: [α]_{D}²⁰=−180.4° (c=0.2, ethanol)

### B5. N-[(1R,2R)-2-(3,4-Dimethoxyphenyl)cyclohexyl]-4-benzoylbenzamide

4.0 g of (1R,2R)-2-(3,4-dimethoxyphenyl)cyclohexylamine (compound C1) are dissolved in 40 ml of methylene chloride and 10.0 ml of triethylamine. A solution of 4.9 g of benzophenone-4-carbonyl chloride in 100 ml of methylene chloride is added dropwise at RT and the mixture is extracted, after stirring overnight, with 50 ml each of water, 2N hydrochloric acid, satd. sodium hydrogencarbonate solution and water again. The organic phase is dried using sodium sulfate and concentrated. 7.78 g of the title compound are obtained as a crystallizing oil.

M.p.: 119-122.5° C.

Optical rotation: [α]_{D}²⁰=−151.7° (c=0.2, ethanol)

### B8. N-[(1R,2R)-2-(3,4-Dimethoxyphenyl)cyclohexyl]-3-acetobenzamide

Compound B8 is prepared from compound C1 analogously as described in Example B5. Solidifying oil.

Optical Rotation: [α]_{D}²⁰=−127.1° (c=0.2, ethanol)

### C1. (1R,2R)-2-(3,4-Dimethoxyphenyl)-cyclohexylamine

12.0 g of a racemic mixture of (1R,2R)-2-(3,4-dimethoxyphenyl)-cyclohexylamine and (1S,2S)-2-(3,4-dimethoxyphenyl)-cyclohexylamine and 6.2 g of (−)-mandelic acid are dissolved in 420 ml of dioxane and 60 ml of tetrahydrofuran and the solution is stirred overnight at RT. The solid is filtered off with suction, dried, treated with 100 ml of saturated sodium hydrogencarbonate solution and extracted with ethyl acetate. The organic phase is dried using sodium sulfate and concentrated under reduced pressure. 4.8 g of the title compound are obtained of m.p.: 80-18.5° C.

Specific rotation: [α]_{D}²⁰=−58.5° C. (c 1, ethanol).

### D1. Racemic mixture of (1R,2R)-2-(3,4-dimethoxyphenyl)-cyclohexylamine and (1S,2S)-2-(3,4-dimethoxyphenyl)-cyclohexylamine

125 g of a racemic mixture of 1,2-dimethoxy-4-((1R,2R)-2-nitrocyclohexyl)benzene and 1,2-dimethoxy-4-((1S,2S)-2-nitrocyclohexyl)benzene and 120 g of zinc powder or granules are suspended in 1300 ml of ethanol. 220 ml of acetic acid are added dropwise at boiling heat. The precipitate is filtered off with suction and washed with ethanol, and the filtrate is concentrated under reduced pressure. The residue is taken up in hydrochloric acid and extracted with toluene. The aqueous phase is rendered alkaline using 50% strength sodium hydroxide solution, the precipitate is filtered off with suction and the filtrate is extracted with toluene. The organic phase is dried using sodium sulfate and concentrated. 98 g of the title compound are obtained as a crystallizing oil.

Alternatively:

8.5 g of a racemic mixture of 1,2-dimethoxy-4-((1R,2R)-2-nitrocyclohexyl)benzene and 1,2-dimethoxy-4-((1S,2S)-2-nitrocyclohexyl)benzene are dissolved in 400 ml of methanol and treated at RT with 7 ml of hydrazine hydrate and 2.5 g of Raney nickel in portions in the course of 8 h. After stirring overnight at RT, the reaction mixture is filtered, the filtrate is concentrated and the residue is chromatographed on silica gel using a mixture of toluene/ethyl acetate/triethylamine=4/2/0.5. The title compound is obtained as an oil.

### E1. Racemic mixture of 1,2-dimethoxy-4-((1R,2R)-2-nitrocyclohexyl)benzene and 1,2-dimethoxy-4-(1S,2S)-2-nitrocyclohexyl)benzene

8.4 g of a racemic mixture of 1,2-dimethoxy-4-((1R,2R)-2-nitrocyclohex-4-enyl)benzene and 1,2-dimethoxy-4-((1S,2S)-2-nitrocyclohex-4-enyl)benzene are dissolved in 450 ml of methanol, treated with 2 ml of conc. hydrochloric acid and hydrogenated after addition of 500 mg of 10% strength Pd/C. The reaction mixture is filtered and the filtrate is concentrated. M.p.: 84-86.5° C.

### F1. Racemic mixture of 1,2-dimethoxy-4-((1R,2R)-2-nitrocyclohex-4-enyl)benzene and 1,2-dimethoxy-4-(1S,2S)-2-nitrocyclohex-4-enyl)benzene

10.0 g of a racemic mixture of 1,2-dimethoxy-4-((1R,2S)-2-nitrocyclohex-4-enyl)benzene and 1,2-dimethoxy-4-((1S,2R)-2-nitrocyclohex-4-enyl)benzene and 20.0 g of potassium hydroxide are dissolved in 150 ml of ethanol and 35 ml of dimethylformamide. A solution of 17.5 ml of conc. sulfuric acid in 60 ml of ethanol is then added dropwise such that the internal temperature does not exceed 4° C. After stirring for 1 h, the mixture is added to 1 l of ice water, the precipitate is filtered off with suction, washed with water and dried, and the crude product is recrystallized from ethanol. 8.6 g of the title compound of m.p. 82.5-84° C. are obtained.

### G1. Racemic mixture of 1,2-dimethoxy-4-((1R,2R)-2-nitrocyclohex-4-enyl)benzene and 1,2-dimethoxy-4-(1S,2S)-2-nitrocyclohex-4-enyl)benzene

50.0 g of 3,4-dimethoxy-ω-nitrostyrene (compound H1) and 1.0 g (9.1 mmol) of hydroquinone are suspended in 200 ml of dry toluene and treated at −70° C. with 55.0 g (1.02 mol) of liquid 1,3-butadiene. The mixture is stirred at 160° C. for 6 days in an autoclave and then cooled. Some of the solvent is removed on a rotary evaporator, and the resulting precipitate is filtered off with suction and recrystallized in ethanol. M.p.: 113.5-115.50° C.

### H1. 3,4-Dimethoxy-ω-nitrostyrene

207.0 g of 3,4-dimethoxybenzaldehyde, 100.0 g of ammonium acetate and 125 ml of nitromethane are heated to boiling for 3-4 h in 1.0 l of glacial acetic acid. After cooling in an ice bath, the precipitate is filtered off with suction, rinsed with glacial acetic acid and petroleum ether and dried. M.p.: 140-141° C. Yield: 179.0 g.

Commercial Utility

The compounds according to the invention have useful pharmacological properties which make them industrially utilizable. As selective cyclic nucleotide phosphodiesterase (PDE) inhibitors (specifically of type 4), they are suitable on the one hand as bronchial therapeutics (for the treatment of airway obstructions an account of their dilating action but also on account of their respiratory rate- or respiratory drive-increasing action) and for the removal of erectile dysfunction on account of their vascular dilating action, but on the other hand especially for the treatment of disorders, in particular of an inflammatory nature, e.g. of the airways (asthma prophylaxis), of the skin, of the intestine, of the eyes, of the CNS and of the joints, which are mediated by mediators such as histamine, PAF (platelet-activating factor), arachidonic acid derivatives such as leukotrienes and prostaglandins, cytokines, interieukins, chemokines, alpha-, beta- and gamma-interferon, tumor necrosis factor (TNF) or oxygen free radicals and proteases. In this context, the compounds according to the invention are distinguished by a low toxicity, a good enteral absorption (high bioavailability), a large therapeutic breadth and the absence of significant side effects.

On account of their PDE-inhibiting properties, the compounds according to the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negatve sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps, but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus, diabetes mellitus, leukaemia, osteoporosis and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia; as well as for enhancing cognition.

The invention further relates to a method for the treatment of mammals, including humans, which are suffering from one of the above mentioned illnesses. The method is characterized in that a therapeutically active and pharmacologically effective and tolerable amount of one or more of the compounds according to the invention is administered to the ill mammal.

The invention further relates to the compounds according to the invention for use in the treatment and/or prophylaxis of illnesses, especially the illnesses mentioned.

The invention further relates to the compounds according to the invention having PDE, particularly PDE4, inhibiting properties.

The invention also relates to the use of the compounds according to the invention for the production of pharmaceutical compositions which are employed for the treatment and/or prophylaxis of the illnesses mentioned.

The invention furthermore relates to pharmaceutical compositions for the treatment and/or prophylaxis of the illnesses mentioned, which contain one or more of the compounds according to the invention.

Additionally, the invention relates to an article of manufacture, which comprises packaging material and a pharmaceutical agent contained within said packaging material, wherein the pharmaceutical agent is therapeutically effective for antagonizing the effects of the cyclic nucleotide phosphodiesterase of type 4 (PDE4), ameliorating the symptoms of an PDE4-mediated disorder, and wherein the packaging material comprises a label or package insert which indicates that the pharmaceutical agent is useful for preventing or treating PDE4-mediated disorders, and wherein said pharmaceutical agent comprises one or more compounds of formula 1 according to the invention. The packaging material, label and package insert otherwise parallel or resemble what is generally regarded as standard packaging material, labels and package inserts for pharmaceuticals having related utilities.

The pharmaceutical compositions are prepared by processes which are known per se and familiar to the person skilled in the art. As pharmaceutical compositions, the compounds according to the invention (=active compounds) are either employed as such, or preferably in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries or excipients which are suitable for the desired pharmaceutical formulations on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

The administration of the pharmaceutical compositions according to the invention may be performed in any of the generally accepted modes of administration available in the art. Illustrative examples of suitable modes of administration include intravenous, oral, nasal, parenteral, topical, transdermal and rectal delivery. Oral delivery is preferred.

For the treatment of disorders of the respiratory tract, the compounds according to the invention are preferably also administered by inhalation in the form of an aerosol; the aerosol particles of solid, liquid or mixed composition preferably having a diameter of 0.5 to 10 μm, advantageously of 2 to 6 μm.

Aerosol generation can be carried out, for example, by pressure-driven Jet atomizers or ultrasonic atomizers, but advantageously by propellant-driven metered aerosols or propellant-free administration of micronized active compounds from inhalation capsules.

Depending on the inhaler system used, in addition to the active compounds the administration forms additionally contain the required excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of apparatuses are available with which aerosols of optimum particle size can be generated and administered, using an inhalation technique which is as right as possible for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhaler described in European Patent Application EP 0 505 321), using which an optimal administration of active compound can be achieved.

For the treatment of dermatoses, the compounds according to the invention are in particular administered in the form of those pharmaceutical compositions which are suitable for topical application. For the production of the pharmaceutical compositions, the compounds according to the invention (=active compounds) are preferably mixed with suitable pharmaceutical auxiliaries and further processed to give suitable pharmaceutical formulations. Suitable pharmaceutical formulations are, for example, powders, emulsions, suspensions, sprays, oils, ointments, fatly ointments, creams, pastes, gels or solutions.

The pharmaceutical compositions according to the invention are prepared by processes known per se. The dosage of the active compounds is carried out in the order of magnitude customary for PDE inhibitors. Topical application forms (such as ointments) for the treatment of dermatoses thus contain the active compounds in a concentration of, for example, 0.1-99%. The dose for administration by inhalation is customarly between 0.1 and 3 mg per day. The customary dose in the case of systemic therapy (p.o. or i.v.) is between 0.03 and 3 mg/kg per day.

Biological Investigations

The second messenger cyclic AMP (CAMP) is well-known for inhibiting inflammatory and immunocompetent cells. The PDE4 isoenzyme is broadly expressed in cells involved in the initiation and propagation of inflammatory diseases (H Tenor and C Schudt, in “Phosphodiesterase inhibitors”, 21-40, “The Handbook of Immunopharmacology”, Academic Press, 1996), and its inhibition leads to an increase of the intracellular CAMP concentration and thus to the inhibition of cellular activation (J E Souness et al., Immunopharmacology 47: 127-162, 2000).

The antiinflammatory potential of PDE4 inhibitors in vivo in various animal models has been described (MM Teixeira, TIPS 18: 164-170, 1997). For the investigation of PDE4 inhibition on the cellular level (in vitro), a large variety of proinflammatory responses can be measured. Examples are the superoxide production of neutrophilic (C Schudt et al., Arch Pharmacol 344: 682-690, 1991) or eosinophilic (A Hatzelmann et al., Brit J Pharmacol 114: 821-831, 1995) granulocytes, which can be measured as luminol-enhanced chemiluminescence, or the synthesis of tumor necrosis factor-α in monocytes, macrophages or dendritic cells (Gantner et al., Brit J Pharmacol 121: 221-231, 1997, and Pulmonary Pharmacol Therap 12: 377-386, 1999). In addition, the immunomodulatory potential of PDE4 inhibitors is evident from the inhibition of T-cell responses like cytokine synthesis or proliferation (D M Essayan, Biochem Pharmacol 57: 965-973, 1999). Substances which inhibit the secretion of the afore-mentioned proinflammatory mediators are those which inhibit PDE4. PDE4 inhibition by the compounds according to the invention is thus a central indicator for the suppression of inflammatory processes.

Method for Measuring Inhibition of PDE4 Activity

PDE4 activity was determined as described by Thompson et al. (Adv Cycl Nucl Res 10: 69-92, 1979) with some modifications (Bauer and Schwabe, Naunyn-Schmiedeberg's Arch Pharmacol 311: 193-198, 1980). At a final assay volume of 200 μl (96 well microtiter plates) the assay mixture contained 20 mM Tris (pH 7.4), 5 mM MgCl₂, 0.5 μM cAMP, [³H]cAMP (about 30,000 cpm/assay), the test compound and an aliquot of cytosol from human neutrophils which mainly contains PDE4 activity as described by Schudt et al. (Naunyn-Schmiedeberg's Arch Pharmacol 344: 682-690, 1991); the PDE3-specific inhibitor Motapizone (1 μM) was included to suppress PDE3 activity originating from contaminating platelets. Serial dilutions of the compounds were prepared in DMSO and further diluted 1:100 (v/v) in the assays to obtain the desired final concentrations of the inhibitors at a DMSO concentration of 1% (v/v) which by itself only slightly affected PDE4 activity.

After preincubation for 5 min at 37° C., the reaction was started by the addition of substrate (cAMP) and the assays were incubated for further 15 min at 37° C. 50 μl of 0.2 N HCl was added to stop the reaction and the assays were left on ice for about 10 min. Following incubation with 25 μg 5′-nucleotidase (Crotalus atrox snake venom) for 10 min at 37° C., the assays were loaded on QAE Sephadex A-25 (1 ml bed volume). The columns were eluted with 2 ml of 30 mM ammonium formiate (pH 6.0) and the eluate was counted for radioactivity. Results were corrected for blank values (measured in the presence of denatured protein) which were below 5% of total radioactivity. The amount of cyclic nucleotides hydrolyzed did not exceed 30% of the original substrate concentration. The IC₅₀-values for the compounds according to the invention for the inhibition of the PDE4 activity were determined from the concentration-inhibition curves by nonlinear-regression.

Representative inhibitory values determined for the compounds according to the invention follow from the following table A, in which the numbers of the compounds correspond to the numbers of the examples.

TABLE A
Inhibition of the PDE4 activity

| Compound | −log IC₅₀ |
|---|---|
| 1 | 8.62 |
| 2 | 8.61 |
| 3 | 8.71 |
| 4 | 8.95 |
| 6 | 8.64 |
| 7 | 9.38 |
| 37 | 8.75 |

1. Compounds of the formula I, 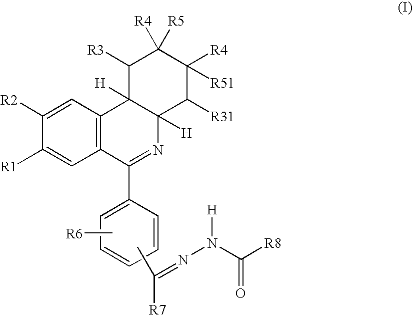
in which
R1 is hydroxyl, 1-4C-alkoxy, 3-7C-cycloalkoxy, 3-7C-cycloalkylmethoxy or completely or predominantly fluorine-substituted 1-4C-alkoxy,
R2 is hydroxyl, 1-4C-alkoxy, 3-7C-cycloalkoxy, 3-7C-cycloalkylmethoxy or completely or predominantly fluorine-substituted 1-4C-alkoxy,
or in which R1 and R2 together are a 1-2C-alkylenedioxy group,
R3 is hydrogen or 1-4C-alkyl,
R31 is hydrogen or 1-4C-alkyl,
or in which R3 and R31 together are a 1-4C alkylene group,
R4 is hydrogen or 1-4C-alkyl,
R5 is hydrogen,
R51 is hydrogen,
or in which R5 and R51 together represent an additional bond,
R6 is hydrogen, halogen, nitro, 1-4C-alkyl, trifluoromethyl or 1-4-C-alkoxy,
R7 is 1-4C-alkyl, 3-7C-cycloalkyl, 3-7C-cycloalkylmethyl, pyridinyl, phenyl or R71- and/or R72-substituted phenyl, wherein
R71 is halogen, hydroxyl, cyano, trifluoromethyl, carboxyl, nitro, 1-4C-alkyl or 1-4C-alkoxy,
R72 is 1-4C-alkoxy,
R8 is 1-4C-alkyl, hydroxy-1-4C-alkyl, 1-4C-alkoxy-1-4C-alkyl, amino, C(O)N(H)R9, phenyl, HetA, aryl-1-4C-alkyl, HetB-1-4C-alkyl, cyano-1-4C-alkyl, 1-4C-alkoxycarbonyl-1-4C-alkyl or R14- and/or R15- and/or R16-substituted phenyl, wherein
R9 is hydrogen, phenyl or R91- and/or R92-substituted phenyl, wherein
R91 is halogen, 1-4C-alkyl, 1-4C-alkoxy, nitro, trifluoromethyl or completely or predominantly fluorine-substituted 1-4C-alkoxy,
R92 is halogen or 1-4C-alkoxy,
HetA is an unsubstituted or R10- and/or R11-substituted heteroaryl radical which is selected from the group consisting of pyrrolyl, furanyl, thiophenyl, pyrazolyl, imidazolyl, oxazolyl, isoxazolyl, thiazolyl, isothiazolyl, thiadiazolyl, pyridinyl, pyrimidinyl, pyrazinyl and pyridazinyl, wherein
R10 is 1-4C-alkyl, phenyl, halogen or trifluoromethyl,
R11 is 1-4C-alkyl,
aryl is phenyl or R12- and/or R13-substituted phenyl, wherein
R12 is 1-4C-alkyl, 1-4C-alkoxy, halogen, nitro or hydroxyl,
R13 is 1-4C-alkoxy or halogen,
HetB is an unsubstituted or R12- and/or R13-substituted indolyl radical,
R14 is 1-4C-alkyl, trifluoromethyl, 1-4C-alkoxy, halogen, nitro, hydroxyl, amino, mono- or di-1-4C-alkylamino or completely or predominantly fluorine-substituted 1-4C-alkoxy,
R15 is 1-4C-alkyl, 1-4C-alkoxy, halogen or completely or predominantly fluorine-substituted 1-4C-alkoxy,
R16 is 1-4C-alkoxy,
and the salts and the E/Z isomers of these compounds.

2. Compounds of the formula I as claimed in claim 1, in which
R1 is 1-2C-alkoxy, 3-5C-cycloalkoxy, 3-5C-cycloalkylmethoxy or completely or predominantly fluorine-substituted 1-2C-alkoxy,
R2 is 1-2C-alkoxy, 3-5C-cycloalkoxy, 3-5C-cycloalkylmethoxy or completely or predominantly fluorine-substituted 1-2C-alkoxy,
R3 is hydrogen,
R31 is hydrogen,
R4 is hydrogen or 1-2C-alkyl,
R5 is hydrogen,
R51 is hydrogen,
or in which R5 and R51 together represent an additional bond,
R6 is hydrogen,
R7 is 1-4C-alkyl or phenyl, R8 is 1-4C-alkyl, hydroxy-2-4C-alkyl, amino, C(O)N(H)R9, phenyl, HetA, aryl-1-2C-alkyl, HetB-1-2C-alkyl, cyano-1-2C-alkyl, 1-4C-alkoxycarbonyl-1-2C-alkyl or R14- and/or R15- and/or R16-substituted phenyl, wherein
R9 is hydrogen, phenyl or R91-substituted phenyl, wherein
R91 is halogen,
HetA is an unsubstituted or R10-substituted heteroaryl radical which is selected from the group consisting of pyrrolyl, furanyl, pyrazolyl, thiadiazolyl and pyridinyl, wherein
R10 is 1-4C-alkyl,
aryl is phenyl or R12- and/or R13-substituted phenyl, wherein
R12 is 1-4C-alkoxy,
R13 is 1-4C-alkoxy,
HetB is an unsubstituted or R12- and/or R13-substituted indol-3-yl radical,
R14 is 1-4C-alkyl, trifluoromethyl, 1-4C-alkoxy, halogen, nitro, hydroxyl, amino, mono- or di-1-4C-alkylamino or completely or predominantly fluorine-substituted 1-2C-alkoxy,
R15 is 1-4C-alkoxy or completely or predominantly fluorine-substituted 1-2C-alkoxy,
R16 is 1-4C-alkoxy,
and the salts and the E/Z isomers of these compounds.

3. Compounds of the formula I as claimed in claim 1, in which
R1 is methoxy,
R2 is methoxy,
R3, R31, R4, R5 and R51 are hydrogen,
R6 is hydrogen,
R7 is methyl or phenyl,
R8 is methyl, hydroxypropyl, amino, C(O)N(H)R9, phenyl, HetA, arylmethyl, indol-3-ylmethyl, cyanomethyl, ethoxycarbonylmethyl or R14- and/or R15- and/or R16-substituted phenyl, wherein
R9 is hydrogen or R91 -substituted phenyl, wherein
R91 is fluorine,
HetA is an unsubstituted furanyl, pyrrolyl or pyridinyl radical, a R10-substituted thiadiazolyl radical or a R10substituted pyrazolyl radical, wherein
R10 is methyl,
aryl is phenyl or R12- and/or R13-substituted phenyl, wherein
R12 is methoxy,
R13 is methoxy,
R14 is methyl, trifluoromethyl, methoxy, fluoro, chloro, nitro, hydroxyl, amino or dimethylamino,
R15 is methoxy,
R16 is methoxy,
and the salts and the E/Z isomers of these compounds.

4. Compounds of the formula I as claimed in claim 1, in which either
R1 is methoxy,
R2 is methoxy,
R3, R31, R4, R5 and R51 are hydrogen,
R6 is hydrogen,
R7 is methyl,
R8 is methyl, hydroxypropyl, amino, C(O)N(H)R9, phenyl, HetA, 3-methoxyphenyl, 4-chlorophenyl, 4-nitrophenyl, 4-hydroxyphenyl, 4-aminophenyl, 4-methylphenyl, 3-chlorophenyl, 3-nitrophenyl, 3,4-dimethoxyphenyl, 3,4,5-trimethoxyphenyl, 4-dimethylaminophenyl, 4-methoxyphenyl, 4-trifluoromethylphenyl, 4-fluorophenyl, arylmethyl, indol-3-ylmethyl, cyanomethyl or ethoxycarbonylmethyl, wherein
R9 is hydrogen or 4-fluorophenyl,
HetA is 1H-pyrrol-2-yl, pyridin-4-yl, furan-2-yl, pyridin-3-yl, 3-methyl-1H-pyrazol-5-yl or 4-methyl[1,2,3]thiadiazol-5-yl,
aryl is phenyl, 4-methoxyphenyl or 3,4-dimethoxyphenyl,
or
R1 is methoxy,
R2 is methoxy,
R3, R31, R4, R5 and R51 are hydrogen,
R8 is hydrogen,
R7 is phenyl,
R8 is methyl or amino,
and the salts and the E/Z isomers of these compounds.

5. Compounds of the formula I according to either claim 1, 2, 3 or 4 in which the hydrogen atoms in positions 4a and 10b are in the cis position relative to one another, and the salts and the E/Z isomers of these compounds.

6. Compounds of the formula I according to either claim 1, 2, 3 or 4 which have with respect to the positions 4a and 10 b the configuration shown in formula I★: 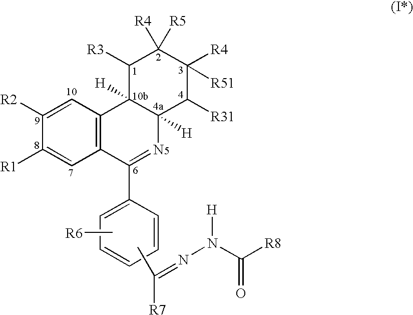
and the salts and the E/Z isomers of these compounds.

7. Compounds of the formula I as claimed in claim 1 for use in the treatment or prevention of diseases.

8. A pharmaceutical composition comprising one or more compounds of the formula I as claimed in claim 1 together with customary pharmaceutical excipients and/or vehicles.

9. The use of compounds of the formula I as claimed in claim 1 and/or their pharmacologically acceptable salts for the production of pharmaceutical compositions for treating or preventing respiratory disorders and/or dermatoses.

10. A method for treating illnesses in a patient comprising administering to said patent a therapeutically effective amount of a compound of the formula I as claimed in claim 1.

11. A method for treating airway disorders in a patient comprising administering to said patient a therapeutically effective amount of a compound of the formula I as claimed in claim 1.
